(19) United States
(12) Patent Application Publication
(10) Pub. No.: US 2020/0368044 A1
(43) Pub. Date: Nov. 26, 2020

(54) PROSTHETIC SOCKET CASTING CYLINDER AND METHOD

(71) Applicant: PC3 Innovations, LLC, Missoula, MT (US)

(72) Inventors: Randy S. Rosenquist, Missoula, MT (US); Clifford H. Creekmore, Stevensville, MT (US)

(21) Appl. No.: 16/422,577

(22) Filed: May 24, 2019

(51) Int. Cl. A61F 2/80; A61F 2/76; A61F 2/78

(52) U.S. Cl. CPC A61F 2/80; A61F 2/76; A61F 2002/5012; A61F 2002/807; A61F 2002/805; A61F 2/7843

(57) ABSTRACT

A prosthetic socket casting cylinder and method for forming a prosthetic limb socket about a distal end portion of residual limb, while the residual limb is weight bearing, the prosthetic socket casting cylinder providing a height adjustable cylinder carrying an internal height adjustable distal interface socket, a pneumatic supply for pressurizing an internal chamber of the cylinder, plural weight sensors for sensing and determining downward pressure and weight exerted upon the cylinder, and a controller for vertically adjustably positioning the cylinder and distal interface, and to determine ratios of downward pressure/weight exerted upon the distal interface and cylinder.

### TECHNICAL FIELD

This invention relates to prosthetic devices, and more particularly to a prosthetic socket casting cylinder and a method for casting a prosthetic socket about a residual limb, while the residual limb is weight bearing.

### BACKGROUND OF THE INVENTION

Prosthetic limbs are generally attached to a user's residual limb by means of a socket that extends over and about a distal end and surrounding portions of the residual limb. Prosthetic sockets were originally hand carved from wood or bone and were cushioned with leather pads. The comfort and functionality of such hand carved prosthetic sockets was very much dependent upon the skill and experience of the person carving the socket, and such sockets were frequently uncomfortable, expensive and difficult to reproduce/replace when the sockets wore out. Further, the socket utility and comfort was dependent upon the user's residual limb not changing, such as by losing or gaining weight. Such sockets also tended to concentrate all of the user's weight, that was borne by the prosthetic, onto a small concentrated surface within the socket which led to discomfort.

Technological advances have led to the development of machines for formation of prosthetic sockets. Known apparatus and methods for forming prosthetic sockets typically provide a liner or sock that is placed over the residual limb. The exterior facing surface of the liner/sock is thereafter covered with a casting material, such as, but not limited to, plaster or carbon pre-preg. After the casting material has hardened/cured, which forms a negative mold, the negative mold is removed from the residual limb. The negative mold is thereafter used to form a positive mold which replicates the physical structure, shape, configuration and sizing of the residual limb and distal end thereof.

Originally, the casting material was manually pressed/molded against and about the residual limb, and distal end thereof, by the prosthetist using hand pressure and while the patient was seated. With more recent developments, cylinders have been employed wherein fluidic pressure is exerted against the casting material to uniformly force the casting material against the residual limb and distal end thereof.

As noted previously, known prosthetic sockets tended to concentrate all, or nearly all of the user's weight on a small area (typically the very bottom or distal end) of the residual limb where the residual limb physically contacted the interior surface of the socket. This lead to rub spots, calluses, discomfort and “hot-spots” that lead to infections and worse.

It has been recognized that prosthetic socket functionality and comfort can be increased if the socket distributes the downward force/weight of the user throughout the socket rather than allowing the downward force/weight to be concentrated at the bottom of the socket and at the distal end of the residual limb. This weight distribution is accomplished by accurately forming the socket about the exterior facing surfaces (circumferentially extending surfaces) of the residual limb adjacent to and spacedly adjacent to the distal end of the residual limb. The added surface area distributes the downward forces/weight across a greater surface area and leads to a more comfortable socket. However, accurately forming such a socket to effectively distribute the user's weight and to avoid rub spots and discomfort has proven problematic and difficult.

One recognized drawback to known prosthetic socket casting apparatus and methods is that fluidic pressure inside the cylinder, that is employed to press the casting material uniformly against the residual limb, exerts forces on the residual limb which are generally parallel to a longitudinal axis of the residual limb. In other words, pressure within the cylinder tends to force the residual limb out of the liner and out of the cylinder which results in a mold that is not accurate and tends to be too inwardly tapered or pointed at the mold's most interior end portion. (The distal end of the residual limb).

A further drawback is that sockets are cast while the user's limb is not weight bearing. As a result, the muscle structure, and tissue position of the limb is not oriented as it would be when the limb is weight bearing. The result is that the socket is malformed for the purpose for which it is intended.

A still further drawback is that users of prosthetic limbs tend to favor, or to place more body weight on a remaining natural limb than on a prosthetic limb. Such unequal weight distribution can lead to spinal alignment problems and joint degeneration. It is therefore important to that equal amounts of body weight are exerted on both the prosthetic limb and on the natural limb.

An even still further drawback is that known prosthetic casting apparatus and methods use fluid impermeable liners that have a bottom portion that physically contacts the distal end of the residual limb, and pressure within the cylinder causes the bottom portion of the liner to contractor radially inwardly which tends to “squeeze” the residual limb out of the cylinder.

Another recognized drawback to known prosthetic casting apparatus and methods is that they require external power sources.

Another recognized drawback to known prosthetic casting apparatus and methods is that they exert uncontrolled proximal\upward pressures on the patient's limb which place the patients' skin under tension and results in bony prominences of the limb becoming more pronounced and exposed, ultimately leading to less comfort in the prosthetic socket.

Another recognized drawback of known casting cylinders is that there is too much volume under the patents limb and thus the patient is ejected/lifted from the cylinder when pressure is applied.

What is needed to overcome known/recognized drawbacks in casting cylinders and methods, and other drawbacks not yet known/recognized is a casting cylinder that does not allow casting forces under the structure supporting the residual limb, and a method for using the same.

Our invention overcomes these and other/further drawbacks by interconnecting an internal flexible sleeve to the bottom of the cylinder to control and limit all upward forces exerted on the limb.

Our invention loads the residual limb, in an optimal, controllable and measurable manner and does not create unwanted skin tension and traction.

Our invention supports the residual limb utilizing compression of the soft tissues and interosseous areas between the bones, creating natural unloading of the bony prominences.

Our invention does not require external power or water sources and can be operated utilizing a positive-displacement air pump (such as, but not limited to a bicycle tire pump) and gravity which allows our invention to meet functional and geographical needs and goals and works equally as well in remote or third world countries as it does in an advanced clinic setting.

Our invention utilizes real-time measurable data to adjust settings to specific patient needs.

Our invention monitors bodyweight on the entire system, distal end of limb (critical for prosthetic fit and comfort) and patient feedback as it relates to comfort levels.

Our invention has plural lift assemblies that allow a practitioner to easily customize the casting cylinder to the specific patient requirements because proper orientation of the casting cylinder decreases chances for patient compensation, e.g. poor alignment and weight distribution, resulting in a better cast. The plural lift assemblies reduce fall risk and discomfort to the patient and save time and effort associated with multiple adjustments required in other systems and the plural lift assemblies do not require use of height blocks or stacks to compensate for patient differences.

Our invention provides a casting cylinder apparatus that is self-contained with on-board sensors to display feedback where adjustments can be made to components by integrated controls in real time and that allows prosthetic practitioners to design prosthetic sockets with greater quality of fit, are reproducible and in less time than traditional casting methods so that patients can be ambulating in a diagnostic socket shortly after completing the casting process.

Our invention provides an apparatus and method for measuring multiple data parameters including, but not limited to, bodyweight applied to the cylinder, bodyweight applied to the distal interface, internal and external pressures, chamber compression and internal forces and providing an ability to adjust all these factors to create an impression closest to the final prosthetic socket as possible by loading the patients' residual limb in a calculated, controllable and reproducible way.

Our invention provides a full-length flexible sleeve which, when used in conjunction with the internal center lift assembly, restricts the amount of volume that is under the patent and causes more pressure or force being applied to the wet distal cast. The inner lift assembly restricts the volume and concentrates pressure where needed.

Our invention provides a prosthetic socket casting apparatus and method that overcomes known and unknown drawbacks to current apparatus and methods.

### BRIEF SUMMARY OF THE INVENTION

A first aspect of the present invention relates to a prosthetic socket casting cylinder and method for forming a prosthetic limb socket about a distal end portion of a residual limb, while the residual limb is weight bearing, provides a cylinder defining an interior chamber having a top end portion defining a top orifice surrounded by a top perimeter edge and a bottom end portion, the interior chamber has an interior diameter and an interior depth that extends between the top end portion and the bottom end portion, the cylinder further has an outwardly facing surface and an inwardly facing surface and the cylinder defines plural ports that all communicate between the outwardly facing surface and the interior chamber. A flexible sleeve securing band extends substantially about the outwardly facing surface proximate the top peripheral edge, and a slip flange extends substantially about the outwardly facing surface proximate the bottom end portion. The slip flange defines plural fastener holes. A generally planar cylinder base is carried at the bottom end portion of the cylinder. The cylinder base has opposing top and bottom surfaces, a peripheral edge, an exterior diametric dimension that is larger than an exterior diametric dimension of the cylinder, and the cylinder base defines plural holes for fasteners to secure the cylinder to cylinder base, and the cylinder base defines a hole that communicates with the interior chamber through the cylinder base. A scale is positioned beneath the cylinder base and adjacent the bottom surface thereof to sense downward pressure/weight exerted upon the cylinder, and the scale is positioned upon a cylinder lift assembly. The cylinder lift assembly supports the scale and cylinder and cylinder base and vertically adjustably positions the scale and the cylinder and the cylinder base relative to an underlying supporting surface. The cylinder lift assembly has a generally planar base platform that has a top surface and an opposing bottom surface, and the base platform carries plural spacedly arrayed post supports that are positioned on the top surface of the base platform spacedly adjacent the scale. Each of the plural spacedly arrayed post supports carries a follower bushing that is axially movable along a length of an interconnected threaded vertical adjustment post. Plural spacedly arrayed threaded vertical adjustment posts are carried by the cylinder lift assembly and each of the plural spacedly arrayed threaded vertical adjustment posts has a top end portion and an opposing bottom end portion for contacting the underlying supporting surface. Each of the plural spacedly arrayed threaded vertical adjustment posts communicates with one of the plural spacedly arrayed post supports and the follower bushing carried thereby, and each of the plural spacedly arrayed threaded vertical adjustment posts extends generally perpendicularly to the base platform and axially through the follower bushing. A slave sprocket is threadably interconnected to each of the plural spacedly arrayed threaded vertical adjustment posts so that the slave sprocket rotates thereabout and moves axially along the length of the interconnected threaded vertical adjustment post. A drive sprocket is rotatably carried by the base platform spacedly adjacent one slave sprocket and a drive belt operatively communicates between the drive sprocket and the slave sprocket so that rotation of the drive sprocket is communicated to the slave sprocket by the drive belt. A drive means operatively communicates with the drive sprocket to rotate the drive sprocket and to responsively cause the drive belt to move and to responsively cause the slave sprockets to rotate and to axially move along the length of the interconnected adjustment post to cause the cylinder and the scale and the cylinder base to move vertically relative to the underlying supporting surface. A proximal limiter substantially fluidically seals the top orifice of the interior chamber about the residual limb. A distal interface cup assembly is carried within the interior chamber of the cylinder. The distal interface cup assembly has an upper end portion, a lower end portion, and a platform with an upper surface and an opposing bottom surface. The platform is vertically movable within the interior chamber of the cylinder, and the platform is carried at the upper end portion of the distal interface cup assembly. A distal interface socket defining a concave depression and having a known radius is carried on the upper surface of the platform, and the distal interface socket is defined between plural spaced apart angulated side walls. A vertically expandable/retractable lift means communicates between the bottom surface of the platform and the top surface of the cylinder base within the interior chamber of the cylinder so that the vertically expandable/retractable lift means controllably vertically adjustably moves and positionally maintains the platform and the distal interface socket within the interior chamber of the cylinder at a predetermined desired position. A hole is defined in the platform and the hole communicates between the distal interface socket and the bottom surface of the platform. A load cell is positioned between the distal interface socket and the upper surface of the platform to sense an amount of weight exerted upon the distal interface socket and an inflatable distal interface is carried within the distal interface socket. The distal interface has an upper surface, a bottom surface, an outer circumferential edge and defines a medial hole. The distal interface may be inflatable and may further defined a port for inflow and/or outflow of fluid to inflate/deflate the inflatable distal interface. A tubular flexible sleeve is carried within the interior chamber of the cylinder that extends over and about the distal interface cup assembly and over and about the distal interface, and the flexible sleeve is formed of a resilient, fluid impermeable, elastomeric material and has a top end portion and a bottom end portion and defines a channel extending between the top end portion and the bottom end portion. The flexible sleeve further has an outwardly facing surface and an opposing inwardly facing surface, and the top end portion defines a top peripheral edge that is releasably positionally secured to the top peripheral edge of the cylinder, and the bottom end portion defines a bottom peripheral edge that is releasably positionally secured about the bottom end portion of the cylinder and to the top surface of the cylinder base by the slip flange. An air pump pneumatically communicates with the interior of the cylinder to pressurize the interior chamber between the inwardly facing surface of the cylinder and the outwardly facing surface of the flexible sleeve, and a distal interface air pump pneumatically communicates with the inflatable distal interface to controllably inflate/deflate to the inflatable distal interface. A scale upon the underlying supporting surface adjacent to the base platform measures user weight that is not exerted upon the distal interface socket of the distal interface cup assembly, nor upon the cylinder. An air pressure gauge pneumatically communicates with the interior chamber of the cylinder to monitor air pressure within the interior chamber in the space between the inwardly facing surface and the outwardly facing surface of the flexible sleeve. A controller operatively communicates with the drive means for vertically adjustably positioning the height of the cylinder relative to a predetermined desired position, with the vertically expandable/retractable lift means for vertically positioning the height of the distal interface socket and platform within the interior chamber of the cylinder relative to a predetermined desired position, with the load cell to determine the amount of weight exerted upon the distal interface socket, with the air pump, with the scale, with a bleed valve operatively communicating with the bleed valve port, with a pressure relief valve operatively communicating with the pressure relief port, with an air pressure gauge operatively communicating with an air pressure gauge port, with a distal interface air pump, with an operator display, with pressure adjustment means and with a control panel allow an operator to actively monitor the status and position of the prosthetic socket casting cylinder apparatus and to determine and calculate a ratio of weight exerted on the scale and upon the distal interface socket and upon the cylinder.

A further aspect of the present invention relates to a prosthetic socket casting cylinder and method for forming a prosthetic limb socket about a distal end portion of residual limb, while the residual limb is weight bearing and wherein the proximal limiter releasably engages with the top perimeter edge of the top orifice of the cylinder with a fastener, and the proximal limiter simultaneously releasably engages with an outwardly facing surface of the residual limb spaced apart from a distal end of the residual limb, the proximal limiter further having an outer circumferential surface that is proximate to the top end portion of the cylinder and an inner circumferential surface that is variable in diameter and is proximate to the residual limb.

A still further aspect of the present invention relates to a prosthetic socket casting cylinder and method for forming a prosthetic limb socket about a distal end portion of residual limb, while the residual limb is weight bearing, and further comprising a limb liner that is placed over and about the distal end portion of the patient's limb; and casting material positioned over and about the limb liner, and wherein the casting material consolidates/hardens to form the prosthetic socket.

### BRIEF DESCRIPTION OF THE DRAWINGS

Preferred embodiments of the invention are described, below, with reference to the following accompanying drawings.

FIG. 1 is an orthographic front cross sectional view of the instant invention showing a residual limb within the cylinder interior chamber and exerting downward force upon the distal interface and upon the distal interface cup assembly with the flexible sleeve extending thereabout and also showing a block diagram of the controller and control panel.

FIG. 1A is an enlarged, partial cutaway, cross-sectional view of the slip flange, cylinder base and securement of the flexible sleeve.

FIG. 1B is an enlarged, partial cutaway, cross-sectional view of the interconnection of the flexible sleeve and securing band at the top peripheral edge of the cylinder.

FIG. 2A is a plan view of a first embodiment of an iris-type proximal limiter in a closed/contracted orientation.

FIG. 2B is a plan view of the first embodiment of the iris-type proximal limiter in an open/expanded orientation.

FIG. 2C is a plan view of a second embodiment of a mesh-type proximal limiter in a closed/contracted orientation.

FIG. 2D is a plan view of the second embodiment of the mesh-type proximal limiter in an open/expanded orientation.

FIG. 2E is a plan view of a third embodiment of an arch-plate proximal limiter in a closed/contracted orientation.

FIG. 2F is a plan view of the third embodiment of the arch-plate proximal limiter in an open/expanded orientation.

FIG. 3 is an isometric side and top view of the flexible sleeve.

FIG. 4 is an enlarged orthographic side view of one embodiment of the inflatable distal interface and distal interface cup assembly having a locking assembly.

FIG. 5 is an enlarged orthographic side view of a second embodiment of the distal interface cup assembly employing a pneumatic lift means.

FIG. 6 is an enlarged orthographic side view of a third embodiment of the distal interface cup assembly employing a telescoping lift means.

FIG. 7 is an enlarged orthographic side view of a fourth embodiment of the distal interface cup assembly employing a second version of a telescoping lift means.

FIG. 8 is a perspective side and top view of the instant invention showing the cylinder, the casting cylinder scale and the cylinder lift assembly above and underlying supporting surface.

FIG. 9A is a partial cutaway orthographic side view of the threaded adjustment posts and the drive belt showing the directions of movement.

FIG. 9B is an orthographic plan view of the threaded adjustment posts, the drive belt, the slave sprockets and the drive sprocket.

FIG. 10 is an artistic representation of a perspective top view of the present invention.

FIG. 11 is a greatly simplified representation of the present inventive apparatus and method showing the downward forces being sensed, monitored and adjusted for.

### DETAILED DESCRIPTION OF THE PREFERRED EMBODIMENTS

This disclosure of the invention is submitted in furtherance of the constitutional purposes of the U.S. Patent Laws “to promote the progress of science and useful arts”. (Article 1, Section 8).

For the purposes of this invention, the term “residual limb” means from approximately two inches (2″) above the patella to the end of limb. The term “distal end” means the contour at the bottom of the residual limb.

A prosthetic socket casting cylinder and method for forming a prosthetic limb socket about a distal end portion of residual limb, while the residual limb is weight bearing generally provides a cylinder 15, a cylinder lift assembly 40, a proximal limiter 60, a flexible sleeve 70, a distal interface cup assembly 90, a distal interface 110, an air pump 120, a casting cylinder scale 124, a scale 125 and a controller 128.

The cylinder 15 is generally tubular and defines an interior chamber 16 having a bottom end portion 18 and a top end portion 17 defining a top orifice 21 surrounded by a top peripheral edge 22. The interior chamber 16 has an interior diameter 23 and an interior depth 24 that extends between the top end portion 17 and the bottom end portion 18. The cylinder 15 further has an outwardly facing surface 19 and an inwardly facing surface 20 and the cylinder 15 defines plural ports, such as an air inlet port 27, a pressure relief port 28, a bleed valve port 26 and an air pressure gauge port 29 that all communicate between the outwardly facing surface 19 and the interior chamber 16. A sleeve securing band 25 extends substantially about the outwardly facing surface 19 proximate the top peripheral edge 22, and a slip flange 30 extends substantially about the outwardly facing surface 19 proximate the bottom end portion 18. The slip flange 30 has a radially enlarged ledge portion proximate a bottom portion and defines plural spacedly arrayed fastener holes 32 to carry fasteners 31 therein.

A generally planar cylinder base 33 is releasably carried at the bottom end portion 18 of the cylinder 15. The cylinder base 33 has opposing top 34 and bottom 35 surfaces, a peripheral edge 38, and has an exterior diametric dimension 39 that is larger than an exterior diametric dimension 37 of the cylinder 15. The cylinder base 33 defines plural spacedly arrayed holes for fasteners 31 to extend therethrough to communicate with the fastener holes 32 defined in the slip flange 30 to secure the cylinder 15 to cylinder base 33. The cylinder base 33 further defines at least one hole 36 that communicates with the interior chamber 16.

The cylinder lift assembly 40 communicates with the cylinder base 33 to vertically adjustably position the cylinder base 33, and cylinder 15, relative to an underlying supporting surface 137. The cylinder lift assembly 40 has a generally planar base platform 41 that has a top surface 42 and an opposing bottom surface. A casting cylinder scale 124 is carried on the top surface 42 of the platform 41 and the casting cylinder scale 124 operatively communicates with the controller 128. The casting cylinder scale 124 senses the total weight of the casting cylinder generally, and any downward forces exerted upon the casting cylinder generally during use. The weight sensed by the casting cylinder scale 124 is communicated to the controller 128 to allow an operator to make necessary adjustments as the sensed weight is compared to other sensed weights and measurements as described hereinafter.

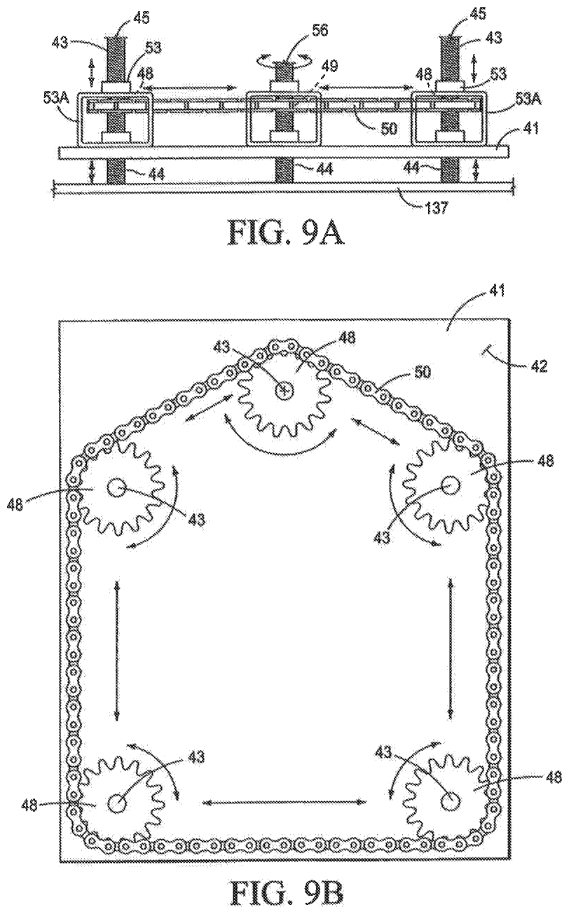

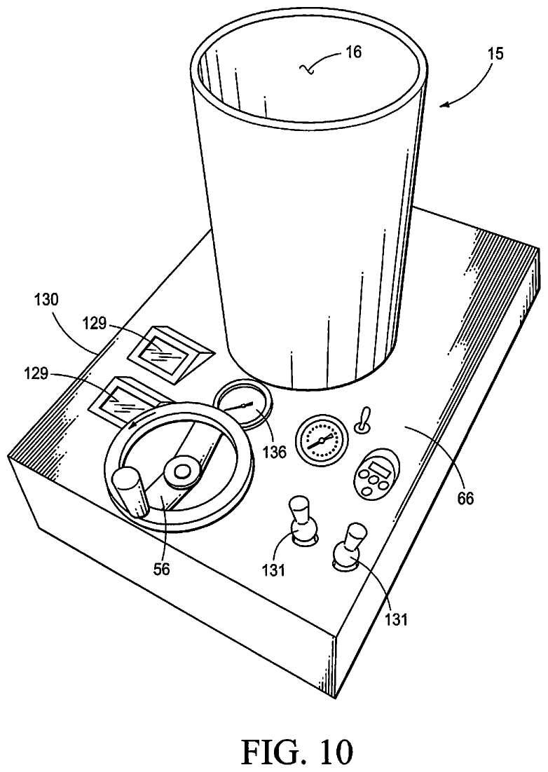

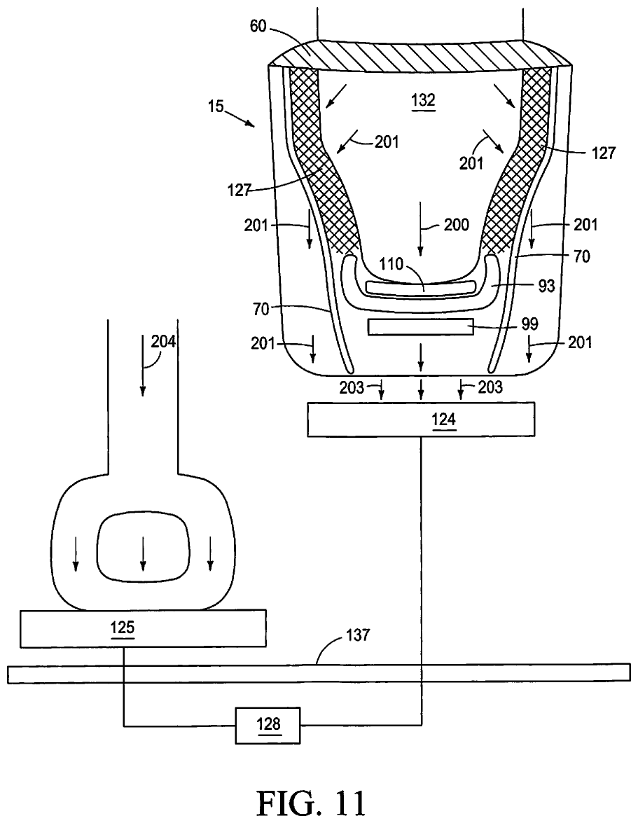

The cylinder lift assembly 40 further carries plural spacedly arrayed post supports 53A that are positioned on the top surface 42 of the base platform 41 spacedly adjacent the casting cylinder scale 124 and each of the plural spacedly arrayed post supports 53A carries a follower bushing 53 that is axially movable along a length of an interconnected threaded vertical adjustment post 43, and each of the plural spacedly arrayed threaded vertical adjustment posts 43 has a top end portion 45 and an opposing bottom end portion 44. Each of the plural spacedly arrayed threaded vertical adjustment posts 43 extends generally perpendicularly to the base platform 41 and axially through the interconnected follower bushing 53. As shown in FIG. 9A, the bottom end portion 44 of each vertical adjustment posts 43 extends through the base platform 41 and through the post support 53A so as to frictionally communicate and rest upon the underlying supporting surface 137. A slave sprocket 48 is threadably carried on each adjustment post 43 and is adjacent to the bushing support 53A so that as the slave sprocket 48 rotates about the adjustment post 43, the bushing support 53A and slave sprocket 48 responsively moves axially along the length of the interconnected threaded adjustment post 43.

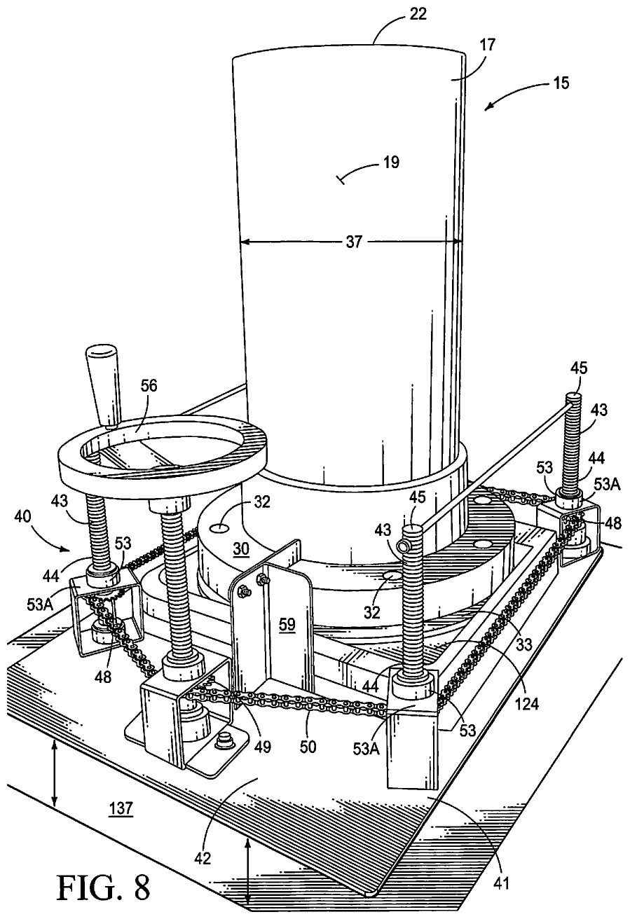

A drive sprocket 49 is rotatably carried by the base platform 41 spacedly adjacent one slave sprocket 48 and a drive belt 50 operatively communicates between the drive sprocket 49 and the spacedly adjacent slave sprocket 48 so that rotation of the drive sprocket 49 is communicated to the slave sprocket 48 by the drive belt 50. A drive means 56, which may be manual (FIG. 8) or may be motor driven (FIG. 9A) operatively communicates with the drive sprocket 49 to rotate the drive sprocket 49 and to responsively cause the drive belt 50 to move and to responsively cause the slave sprockets 48 to rotate and move axially along the interconnected adjustment post 53 which responsively causes the cylinder 15, and casting cylinder scale 124 and cylinder base 33 to move vertically relative to the underlying supporting surface 137.

The drive means 56 may be manual, such as a crank wheel as shown in FIG. 8, or the drive means 56 may be powered, such as, but not limited to, an electric motor that operatively communicates with the controller 128.

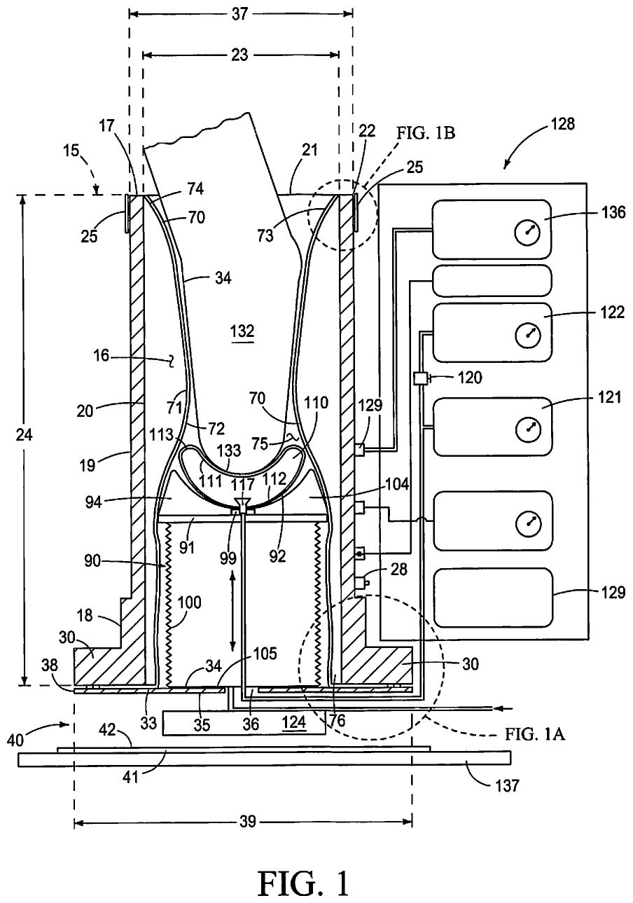

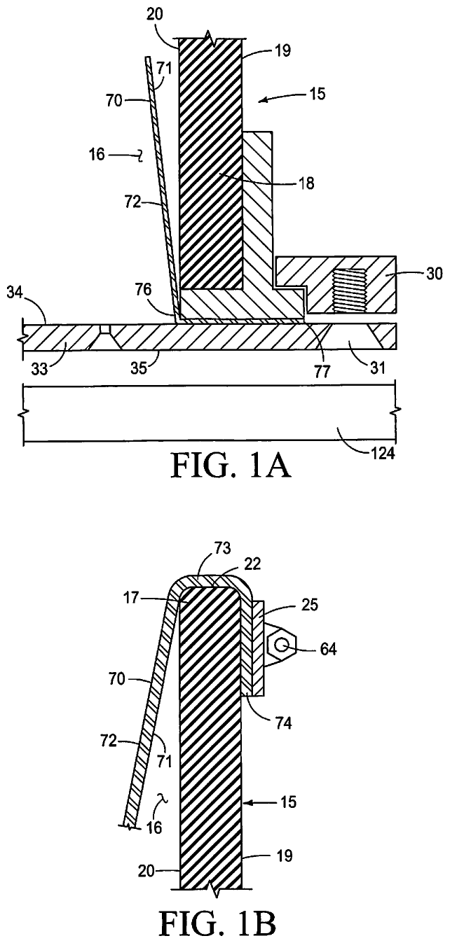

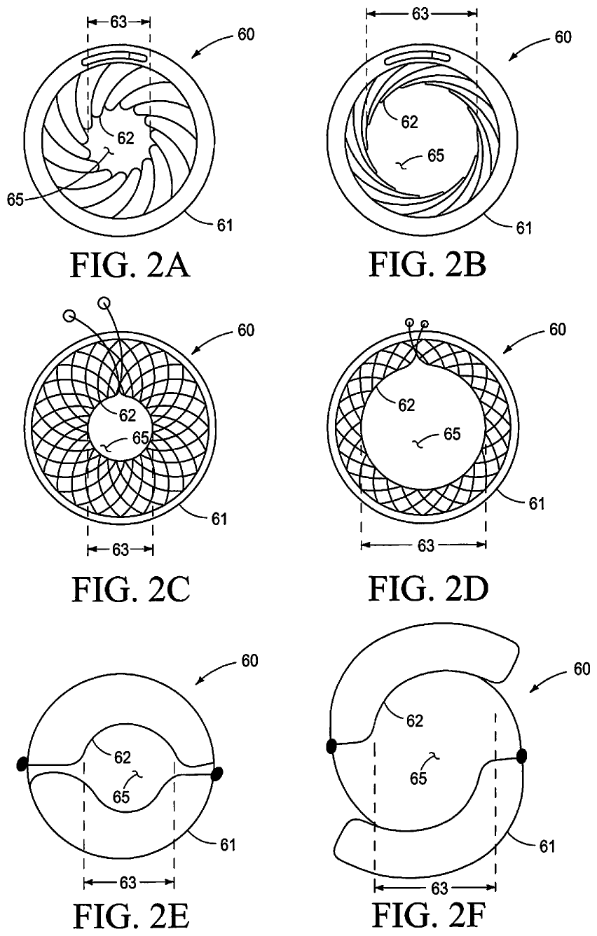

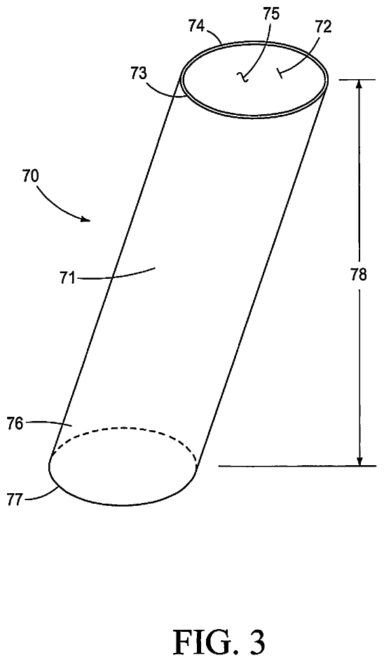

As shown in FIGS. 2A-2F, the proximal limiter 60 is generally circular in peripheral configuration and has an outer circumferential edge 61 and an inner circumferential edge 62 that defines a medial orifice 65. The medial orifice 65 has a variable diameter (See FIGS. 2A-2F) as the proximal limiter 60 is expanded and/or contracted to substantially fluidically seal the top orifice 21 of the interior chamber 16 about the residual limb 132. The outer circumferential edge 61 is releasably engageable with the cylinder 15 by means of the flexible sleeve securing band 25. (FIG. 1B).

The distal interface cup assembly 90 is carried within the interior chamber 16 of the cylinder 15. The distal interface cup assembly 90 has an upper end portion 104 and a lower end portion 105, and has a platform 91 with an upper surface 95a and an opposing bottom surface 95b. The platform 91 is carried at the upper end portion 104 of the distal interface cup assembly 90 and is vertically movable within the interior chamber 16 of the cylinder 15. A distal interface socket 92 which defines a concave depression having a known radius 93 is carried on the upper surface 95a of the platform 91, and the distal interface socket 92 is defined between side walls 94.

A lift means 100, that is vertically extendable/retractable, communicates between the bottom surface 95b of the platform 91 and the top surface 34 of the cylinder base 33 within the interior chamber 16 of the cylinder 15 so that the lift means 100 controllably vertically adjustably moves, and positionally maintains, the platform 91 and the distal interface socket 92 within the interior chamber 16 of the cylinder 15 at a predetermined desired position. A hole 96 is defined in the platform 91 and the hole 96 communicates between the socket 92 and the bottom surface 95b.

A load cell 99 is carried between the distal interface socket 92 and the upper surface 95a of the platform 91 to sense an amount of weight exerted upon the distal interface socket 92 by the residual limb 132. The load cell 99 operatively communicates with the controller 128.

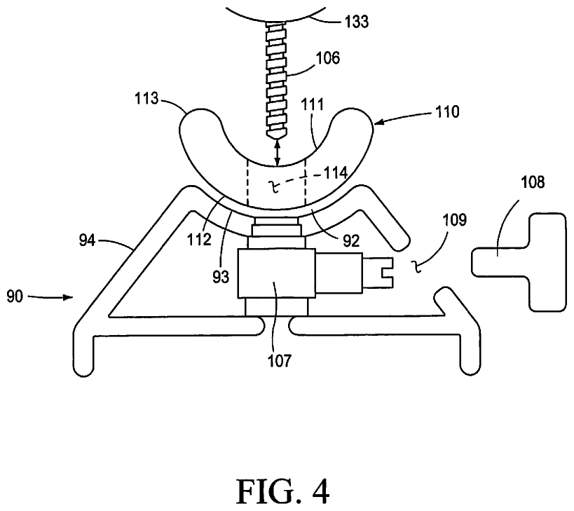

A distal interface 110 is removably positioned within the distal interface socket 92 to provide a spacer and cushion within the distal interface socket 92. The distal interface 110 may have a variety of configurations including, but not limited to, a ball or a flattened ball. The distal interface 110 has an upper surface 111, a bottom surface 112, and an outwardly facing edge 113. As shown in FIG. 4, the distal interface 110 may have the configuration of a doughnut that defines a medial hole 114 therein. Further still, the distal interface 110 may be inflatable and define a port 117 for inflow and/or outflow of fluid or gas to inflate/deflate the inflatable distal interface 110.

The distal interface 110 may be either pneumatic or fluid based and the specific configuration is patient dependent. The size of the distal interface 110 is determined by residual limb circumference. (Type of limb, i.e. tapered, conical or cylindrical, firmness of tissue and condition of limb, e.g. neuromas, bony structure and amputation specifics, will determine the firmness.) The shape of the distal interface 110 is determined by the socket 92 design and suspension methods most advantageous for the patient's needs and goals. A ball or bladder interface is preferable for the cushion liner utilizing suction or elevated vacuum suspension, whereas the doughnut shaped distal interface 110 would be utilized for a locking liner suspension method. (FIG. 4). The doughnut shaped distal interface 110 allows for the locking pin 106 from the locking liner to be engaged and elongation forces to be applied to the limb during the casting process, providing excellent volume, suspension and comfort for the patient.

The distal interface cup assembly 92 is a platform, base or holder for the distal interface 110. Angulation of the sides 94 and radius 93 of the internal arc provide a stable base for the distal interface 110. The design of the distal interface socket 92 produces a conformity between the residual limb, the casting material 127 and the flexible sleeve 70 which creates an accurate distal end of the cast under load. The distal interface cup assembly 90 also resists vertical displacement forces by equalizing the downward vertical bodyweight pressure with the upward vertical pressure from the flexible sleeve 70. (See FIG. 11). The limb is loaded in a controllable, measurable and optimal position for casting, and ultimately ambulation utilizing a prosthesis with enhanced comfort and functionality.

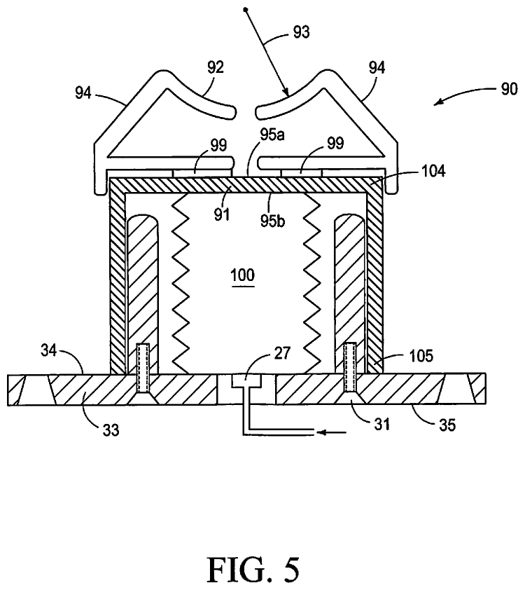

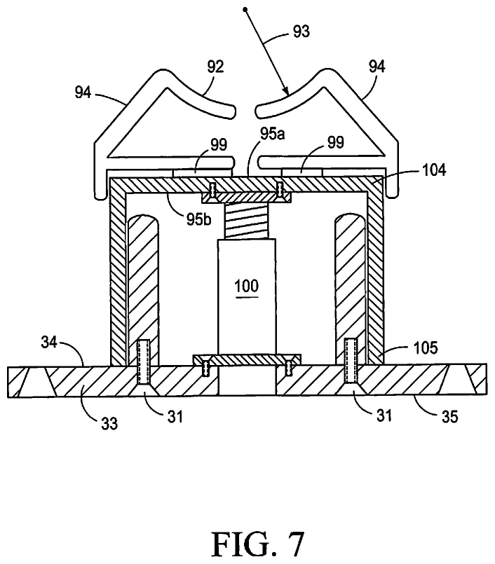

The lift means 100 raises and lowers the distal interface 110 and the distal interface cup assembly 90 within the cylinder 15, providing adjustability for the patient's residual limb length. This optimizes the internal depth 24 of the cylinder 15 to customize for a wide variety of limb lengths. Height adjustment of the lift means 100 is actuated by utilizing the vertically expandable/retractable lift means 100 and is easily activated manually in the case of the threaded mechanism (FIG. 7) or via the control panel 130 for the electric or pneumatic versions. (FIGS. 5, six). The design allows for precise depth of patient in the cylinder 15 under load without the use of bulky spacers or adaptations. The telescopic slide component of the lift means 100 maintains contact with the cylinder 15 and the flexible sleeve 70 which resists compression from the sides within the bottom of the cylinder 15, eliminating upward forces which can push the patient's limb out of the cylinder 15, negatively affecting casting outcome.

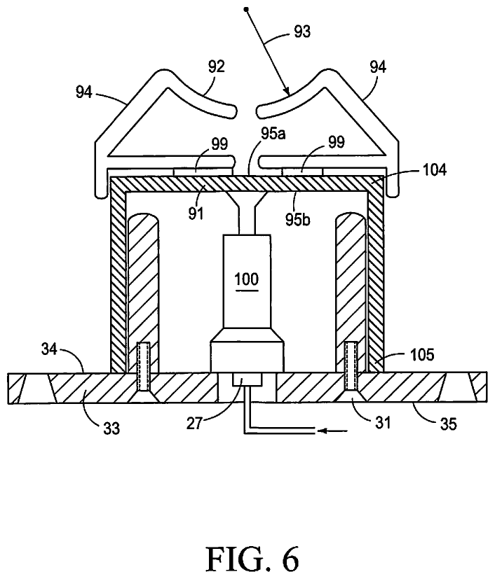

There are three presently contemplated versions of the lift means 100. Air Bladder, either active or passive (FIG. 5), electric piston (FIG. 6) and a threaded elevation unit (FIG. 7).

The cylinder lift assembly 40 raises and lowers the cylinder 15 to correct patient specific height relative to the underlying supporting surface 137. The lift means 100 presets the cylinder 15 to the correct patient specific height. The lift means 100 presets the cylinder 15 to the correct internal depth to coincide with the length of the patient's residual limb, (approximately 2″ above the top of the patella) to allow for adequate tissue containment for prosthetic socket 92 sidewall height. The cylinder lift assembly 40 will lift the entire cylinder 15 to the patient specific height to level hips and match length of sound side lower extremity. The combination of the integrated, adjustable internal and external lift assemblies enables the prosthetic practitioner to customize the casting cylinder 15 to patient requirements, all without adding or subtracting additional componentry.

As shown in FIG. 1, the flexible sleeve 70 is tubular and is carried within the interior chamber 16 of the cylinder 15 and extends over and about the distal interface cup assembly 90, and over and about the distal interface 110 and over and about the residual limb 132. The flexible sleeve 70 is preferably formed of a resilient, fluid impermeable, elastomeric material and has a top end portion 73 and a bottom end portion 76 defines a channel 75 extending between the top end portion 73 and the bottom end portion 76. The flexible sleeve 70 further has a depth 78 and an outwardly facing surface 71 and an opposing inwardly facing surface 72. The top end portion 73 defines a top peripheral edge 74 that is releasably positionally secured to/about the top peripheral edge 22 of the cylinder 15 (FIG. 1B), and the bottom end portion 76 defines a bottom peripheral edge 77 that is releasably positionally secured to/about the bottom end portion 18 of the cylinder 15 by the slip flange 30. (FIG. 1A).

The flexible sleeve 70 is preferably fabricated from a vulcanized or seamless 1/16″ Nitrile Buna-N 40 Duro Rubber Material and has a preferred dimension of approximately 36″×7″″ for a transtibial or below knee socket. The preferred material of the flexible sleeve 70 is high strength with a proven 8× expansion rate and a burst strength of approximately 6500 PSI, yet soft enough to form around the patients' limb during the weight bearing casting process, and the flexible sleeve 70 is designed to expand around the internal components of the apparatus and the patients' residual limb wrapped in the casting material 127. The flexible sleeve 70 inhibits pressure underneath the internal cylinder components, and minimizes upward vertical forces and allowing necessary and natural homeostasis or balance between the bodyweight downward vertical forces and horizontal and upward forces to support the patients' limb in the casting process and ultimately in a well fitted, functional and comfortable prosthetic socket.

The air pump 120, which may be manual or powered, pneumatically communicates with the interior chamber 16 of the cylinder 15 to pressurize the interior chamber 16 between the inwardly facing surface 20 of the cylinder 15 and the outwardly facing surface 72 of the flexible sleeve 70. The air pressure forces casting material 127 about the distal limb 132 to form an anatomically correct/accurate mold.

When an inflatable distal interface 110 is used, a distal interface air pump 121, which may be powered or manual, pneumatically communicates with the inflatable distal interface 110 to controllably inflate/deflate to the inflatable distal interface 110 responsive to operator input.

A scale 125 upon the underlying supporting surface 137 and adjacent to the base platform 41 measures patient body weight that is not exerted upon the distal interface socket 92 of the distal interface cup assembly 90 nor upon the cylinder 15. The scale 125 operatively communicates with the controller 128 so that the operator can measure, determine and monitor what percentages of the user's body weight is being exerted upon which component/scale 124, 125, 99. (See FIG. 11).

An air pressure gauge 136 pneumatically communicates with the interior chamber 16 of the cylinder 15 through a port to monitor air pressure within the interior chamber 16 in the space between the inwardly facing surface 20 of the cylinder 15 and the outwardly facing surface 71 of the flexible sleeve 70. The monitored pressure pneumatically presses the casting material 127 against the external surface 134 of the liner/sock 133 about the residual limb 132 to form an accurate and physiologically correct mold. Active/continuous monitoring of the air pressure and exerted weight is important to ensure the air pressure does not “eject” or tend to “eject” the residual limb 132.

The controller 128, which may be a computer (not shown), operatively communicates with the drive means 56 for vertically adjustably positioning the height of the cylinder 15 relative to a predetermined desired position, with the lift means 100 for vertically positioning the height of the distal interface socket 92 and platform 91 within the interior chamber 16 of the cylinder 15 relative to a predetermined desired position, with the load cell 99 to determine the amount of weight exerted upon the distal interface socket 92, with the air pump 120, with the casting cylinder scale 124, with the scale 125, with a bleed valve, with a pressure relief valve, with the air pressure gauge 136, with the distal interface air pump 121, with an operator display 129, with pressure adjustment means 131 and with a control panel 130 allow an operator to actively monitor the dynamic status, position and conditions about and within of the prosthetic socket casting cylinder apparatus and to determine and calculate a ratio of weight exerted on the scales 124, 125 and on the socket 92. Other monitored measures may include, but not be limited to, temperature, humidity, atmospheric content, etc.

As noted previously, one drawback to known casting cylinders is that application of pneumatic pressure to the residual limb 132 tends to force the residual limb 132 upwardly/out of the cylinder 15 which can result in an imperfect or inaccurate mold. As the pneumatic pressure within the chamber 16 is increased, the ejection forces likewise increase. As shown in FIG. 4, in one preferred embodiment, the locking shaft 106 may be carried on an end portion of a limb liner 133 that is placed upon the residual limb 132. The locking shaft 106 extends through a medial hole 114 defined in the inflatable distal interface 110. The locking shaft 106 extends into a locking base 107 carried below the distal interface socket 92. A locking key 108 is releasably inserted into a hole 109 defined in the side wall 94 of the distal interface cup assembly 90. Engagement of the locking shaft 106 within the locking assembly 107 prevents the tendency of the residual limb 132 from being ejected from the interior chamber 16 when pneumatic pressure is supplied to the interior chamber 16. Although not shown in FIG. 4, the flexible sleeve 70 extends entirely over and about the distal interface cup assembly 90.

As shown in FIG. 5, in a further preferred embodiment, the lift means 100 may be a pneumatic bladder that vertically extends as pneumatic pressure is added, and vertically retracts as pneumatic pressure is released/removed from the bladder.

As shown in FIG. 6, in a further preferred embodiment the lift means 100 is a telescoping cylinder that extends and/or retracts as pneumatic pressure is added/released to the telescoping cylinder.

As shown in FIG. 7, in a still further preferred embodiment, the lift means 100 is a jackscrew that extends and/or retracts as a first element is axially rotated relative to a threadably interconnected second element.

### Operation

The operation of the described apparatus and methodology for implementing the present invention is believed to be readily apparent from the above description, and is briefly summarized at this point.

The present invention would be placed upon an underlying supporting surface 137 that is preferably level and planar.

Patient evaluation is performed including bodyweight, length of residual limb from an upper edge of the socket to distal end. Thorough evaluation of tissue type and coverage as well as identification of any neuromas, sensitive areas, anomalies or areas of concern are noted as usual. Initial loading of distal end of residual using the pneumatic distal interface is conducted to establish pressure tolerance and sizing of the interface. An assistive stabilizer (not shown), such as a walker or parallel bars, are used to provide patient stability and are set to allow safe and unobstructed entry into the cylinder. Once the patient is in position, seated, the initial cylinder settings and calibrations are performed.

The user's total body weight is measured using the scale 125 and the controller 128. The operator would examine the patient, the patient's residual limb 132 and the patient's posture to determine a proper predetermined height for the distal interface socket 94 and the cylinder 15. The user's total body weight is divided in half so that the controller 128 can determine when the correct amount (50%) of the total body weight is exerted on the scale 125 and the remaining half (50%) of the body weight is exerted on a combination of the load cell 99 and the casting cylinder scale 124. The operator further determines how much of the distal limb 132 (length) needs to be molded so as to determine a preferred height of the cylinder 15 relative to the underlying supporting surface 137.

Using the measurements taken earlier the cylinder lift assembly 40 is activated, either manually or from outside power source, to raise entire unit to predetermined height. The distal interface cup assembly 90 is also set to the parameters calculated and determined. A reference mark is located on the patient approximately 2″ above the top of the patella. The patient stands up, using the stabilizer unit for safety, and places the residual limb within the cylinder 15. The top 17 of the cylinder 15 should be at the reference line (not shown) marked on the patient. The patient puts a comfortable amount of weight on the distal interface 110 verifying loading via the interconnected scale 124 and control panel 130. This bodyweight reading assists in determining proper pneumatic pressure within distal interface 110, distal interface tolerance and proper length of internal cylinder and overall cylinder height. Dynamic adjustments to the distal interface 110, cylinder lift assembly 40 and distal interface cup assembly 90 are performed to fine tune the position and to align the upper perimeter 22 of the cylinder 15 with the aforementioned reference mark. The patient removes the limb from the casting cylinder 15 and sits down. Pre-compression of the cylinder 15 is conducted to more easily center the patient within the cylinder 15. The patient's residual limb is wrapped with casting material 127 such as plaster, or carbon pre-preg and the reference mark is transferred, and limb is covered with a plastic bag and placed back into the cylinder 15 and the proximal limiter 60 is secured in place about to the limb. Air pressure is added to the cylinder 15 to a predetermined compression for the specific patient profile while the body weight of the patient is monitored by the scales 124, 125 and the control panel 130. Feedback from the patient is continually monitored. Allow casting material 127 to fully cure, checking the top of the cast material 127, prior to releasing the proximal limiter 60 having the patient withdraw the limb from the cylinder 15.

The drive means 56 is used to adjust to the vertical height of the cylinder lift assembly 40 and the cylinder 15 so as to properly place the top peripheral edge 22 of the cylinder 15 correctly relative to the residual limb a 132 and the patient's body. Using the control panel 132, (or manual adjustments) the vertical height of the distal interface cup assembly platform 91, carrying the distal interface socket 92 is vertically adjustably positioned within the interior chamber 16.

The residual limb 132 is covered with a limb liner 133 so that the casting material 127 is not directly applied to the external surface 134 of the residual limb 132. If the locking assembly 107 type distal interface cup assembly 90 is being used, the locking shaft 106 would extend generally outwardly and downwardly from the distal end portion of the residual limb 132. A supply of casting material 127 would be applied over and about the limb liner 133 covering the distal limb 132. While the casting material 127 is still pliable and moldable, the patient stands upon the scale 125 and places the casting material 127 covered residual limb 132 within the interior chamber 16 of the cylinder 15.

The residual limb 132 is inserted through the medial orifice 65 of the proximal limiter 60 and the distal end portion of the residual limb 132 is placed directly upon the distal interface 110. If a locking limb liner 133 (FIG. 4) is being used, the locking shaft 106 is inserted into the locking base 107. The locking shaft 106 is secured within the locking base with the locking key 108.

The patient stands and exerts downward force simultaneously on both the patient's natural limb (not shown) that is resting upon the scale 125, and also exerting downward force (not shown) on the distal interface socket 94 within the interior chamber 16 of the cylinder 15. The operator, using the control panel 132 that is operatively communicating with the scale 125, with the casting cylinder scale 124 and the load cell 99 verifies that equal amounts of body weight are being exerted on the scale 125 and upon a combination (sum) of the load cell 99 plus the casting cylinder scale 124. The operator may use the distal interface air pump 121 and the air pump 120 to adjust the amount of pressure/weight being exerted upon the distal interface socket 94 and casting cylinder scale 124, and also to adjust height.

The proximal limiter 60 is closed so that the inner circumferential edge 62 thereof is in direct physical contact with the exterior surface 134 of the distal limb 132 so as to provide a pneumatic seal therebetween and the outer circumferential edge 61 is secured to the top peripheral edge 22 of the cylinder 15 by the securing band 25.

The operator, using the control panel 130 (or manually) activates the air pump 120 to cause pneumatic pressure to be added into the interior chamber 16 of the cylinder 15 in the space between the inwardly facing surface 20 of the cylinder 15 and the outwardly facing surface 71 of the flexible sleeve 70. The pneumatic pressure (not shown) causes the flexible sleeve 72 collapse radially inwardly so as to directly frictionally contact the casting material 127 surrounding the limb sleeve 135 and residual limb 132. The pneumatic pressure exerted by the increased air pressure is equal on all exterior surfaces of the residual limb 132. The operator, using the load cell 99 and casting cylinder scale 124 can actively and continuously monitor the amount of downward force/weight being exerted upon the distal interface socket 94, and whether or not the pneumatic pressure exerted upon the residual limb 132 by the pneumatic pressure and air pump 120 is causing the residual limb 130 to be ejected upwardly from the interior chamber 16 of the cylinder 15. By monitoring, and maintaining a consistent amount of weight exerted upon the distal interface socket 94, plus the casting cylinder scale 124 which is optimally about one half of the total body weight of the patient, the operator is able to prevent the pneumatic pressure from deforming the mold about the distal limb 132.

After the casting material 127 has hardened/cured, the residual limb 132 and hardened/cured negative mold is removed from the interior chamber 16 of the cylinder 15 by releasing the threaded shaft 106 from the locking assembly 107 and releasing the proximal limiter 60.

Thereafter, the formed negative mold may be used to form an anatomically correct replica of the distal limb 132 from which a precise prosthetic socket may thereafter be formed. The anatomically correct replica of the distal limb 132 represents the distal limb 132 when the distal limb 132 is weight bearing as it would be when the prosthetic limb is being used by the patient.

Three (3) separate vertical downward forces are exerted, sensed and monitored on the invention during formation of the socket.

The first force 201 (measure #1) is the downward force exerted by the distal/residual bottom end of the limb 132 directly upon the distal interface 110 within the distal interface socket 92 and is sensed by the load cell 99.

The second force 202 (measure #2) is the downward force exerted by the residual limb 132 upon the entire of the cylinder assembly (other than the first force 201 measure #1) which is the weight that is borne by the casting material 127 and the flexible sleeve 70 that is in direct physical contact with the outwardly facing surfaces of the residual limb 132 within the chamber 16 and above the distal interface 110 and below the proximal limiter 60. (Pressure within the chamber 16 and the forces 205 exerted thereby modifies/adjusts the second force 202—less pressure causes a greater first force 200 measure #1 and more pressure lessens the first force 201 measure #1). An object of the present invention and method is to distribute the weight (201, 202) throughout the prosthetic socket to avoid concentration of weight/forces 201, 202 at any single location within the prosthetic socket.

The third force 203 (measure #3) is the total weight of the first force 201 (measure #1) plus the second force 202 (measure #2) plus the weight of the casting cylinder apparatus—and this third force 203 (measure 3) weight is exerted upon and sensed by the casting cylinder scale 124. By subtracting the weight of the casting cylinder apparatus from the third force 203 (measure #3) the instant inventive device and method allows calculation of how much weight the patient is exerting upon the residual limb 132. The optimal amount of weight the patient should exert on the residual limb 132 (201 plus 202) is targeted to be 50% of the patient's total weight, and this calculation is performed by sensing the amount of downward force/weight 204 the patient is exerting upon the remaining limb 206 which is sensed by the scale 125 and while the patient is maintaining proper posture/alignment). Readings from all the weight/force measuring devices 99, 124, 125 are all operatively communicated to the controller 128.

A prosthetic socket casting cylinder and method for forming a prosthetic limb socket about a distal end portion of residual limb, while the residual limb is weight bearing, the prosthetic socket casting cylinder comprising: a cylinder defining an interior chamber having a top end portion, and a bottom end portion, the cylinder further having an outwardly facing surface and an inwardly facing surface and the cylinder defines a port that communicates between the outwardly facing surface and the interior chamber, and a slip flange extends substantially about the outwardly facing surface proximate the bottom end portion; a cylinder base secured to the bottom end portion of the cylinder, the cylinder base having a peripheral edge that has an exterior diametric dimension larger than an exterior diametric dimension of the cylinder; a scale positioned beneath the cylinder base to sense downward pressure/weight exerted upon the cylinder, and the scale is supported upon a cylinder lift assembly; the cylinder lift assembly has, a base platform with opposing top and bottom surfaces and carries plural spacedly arrayed post supports that are positioned on the top surface of the base platform and spacedly adjacent the scale, and each of the post supports carries a follower bushing that is axially movable along a length of the threaded vertical adjustment post, and each threaded vertical adjustment post threadably carries a slave sprocket that is axially rotatably movable along the length of the adjustment post, and each of the plural threaded vertical adjustment posts extend perpendicularly relative to the top and bottom surfaces of the platform and a bottom end portion of each threaded vertical adjustment post engages with the underlying supporting surface, and a drive belt operatively communicating between the slave sprockets so that rotation of one slave sprocket is communicated to a second slave sprocket by the drive belt, and a drive means operatively communicating with the drive belt; a distal interface cup assembly within the interior chamber of the cylinder, and the distal interface cup assembly has, an upper end portion, a lower end portion, and a platform that is vertically movable within the interior chamber, and the platform is carried at the upper end portion of the distal interface cup assembly, and a distal interface socket defining a concave depression in the upper surface of the platform, and a vertically expandable/retractable lift means operatively communicates between the platform and the cylinder base within the interior chamber to controllably vertically adjustably move and positionally maintain the platform within the interior chamber of the cylinder at a predetermined desired position, and a load cell between the socket and the platform to sense an amount of weight exerted upon the socket, and a distal interface within the distal interface socket; a flexible sleeve within the interior chamber of the cylinder that extends over and about the distal interface cup assembly and over and about the distal interface, the flexible sleeve having a top end portion that is positionally anchored to the cylinder, and a bottom end portion that is positionally secure to the cylinder base; an air pump pneumatically communicating with the interior chamber between the inwardly facing surface of the cylinder and an outwardly facing surface of the flexible sleeve; a scale to sense downward pressure/weight that is not exerted upon the distal interface socket; a controller operatively communicating with the drive means, with the vertically expandable/retractable lift means, with the load cell, with the air pump, with the scale, with an operator display, with adjustment means and with a control panel allow an operator to actively monitor the status and position of the prosthetic socket casting cylinder apparatus and to determine and calculate a ratio of weight simultaneously exerted on the scale and on the socket.

A prosthetic socket casting cylinder and method wherein the drive means is a motor.

A prosthetic socket casting cylinder and method wherein the vertically expandable/retractable lift means is a telescoping cylinder.

A prosthetic socket casting cylinder and method d wherein the vertically expandable/retractable lift means is an expandable fluid containing bladder.

A prosthetic socket casting cylinder and method further comprising a drive sprocket rotatably carried by the base platform and operatively communicating with the drive belt so that rotation of the drive sprocket is communicated to the slave sprocket.

A prosthetic socket casting cylinder and method wherein the drive means rotates the drive sprocket which responsively moves the drive belt and which responsively rotates the slave sprockets to cause the cylinder base to move vertically relative to the underlying supporting surface.

A prosthetic socket casting cylinder and method wherein the cylinder is tubular and defines a top orifice surrounded by a top peripheral edge, and has a bottom end portion, and the interior chamber has an interior diameter and an interior depth that extends between the top end portion and the bottom end portion, the tubular cylinder further having an outwardly facing surface and an inwardly facing surface and the tubular cylinder defines an air inlet port, a pressure relief port, a bleed valve port, an air pressure gauge port that all communicate between the outwardly facing surface and the interior chamber.

A prosthetic socket casting cylinder and method further comprising: a sleeve securing band that extends substantially about the outwardly facing surface of the cylinder proximate the top peripheral edge; a slip flange that extends substantially about the outwardly facing surface of the cylinder proximate the bottom end portion, the slip flange defining plural spacedly arrayed fastener holes; and the cylinder base defines plural spacedly arrayed holes for fasteners to extend therethrough to communicate with the plural spacedly arrayed fastener holes defined in the slip flange.

A prosthetic socket casting cylinder and method further comprising: a proximal limiter that substantially fluidically seals the top orifice of the interior chamber.

A prosthetic socket casting cylinder and method wherein the distal interface socket has a known radius and is defined between plural spaced apart angulated side walls that extend from the upper surface of the platform.

A prosthetic socket casting cylinder and method further comprising: a hole is defined in the platform and the hole communicates between the distal interface socket and the bottom surface of the platform.

A prosthetic socket casting cylinder and method wherein the distal interface is inflatable, and the distal interface has an upper surface, a bottom surface, an outer circumferential edge and defines a medial hole, the distal interface further has a port for inflow and/or outflow of fluid to inflate/deflate the inflatable distal interface.

A prosthetic socket casting cylinder and method wherein the flexible sleeve is formed of a resilient, fluid impermeable, elastomeric material and has a top end portion and a bottom end portion and defines a channel extending between the top end portion and the bottom end portion, and the flexible sleeve further has an outwardly facing surface and an opposing inwardly facing surface, and the top end portion defines a top peripheral edge that is releasably secured to the top peripheral edge of the top orifice of the cylinder, and the bottom end portion defines a bottom peripheral edge that is releasably secured about the bottom end portion of the cylinder and to the top surface of the cylinder base.

A prosthetic socket casting cylinder and method wherein the scale is placed upon an underlying supporting surface adjacent to the base platform to simultaneously measure weight that is not exerted upon the distal interface socket of the distal interface cup assembly.

A prosthetic socket casting cylinder and method further comprising: a controller operatively communicating with the drive means for vertically adjustably positioning and maintaining the cylinder relative to a predetermined desired position, with the vertically expandable/retractable lift means for vertically positioning the distal interface socket within the interior chamber of the cylinder relative to a predetermined desired position, with the load cell to determine an amount of weight exerted upon the distal interface socket, with the air pump, with the scale, with measuring instruments operatively communicating with the plural ports defined in the cylinder, with a distal interface air pump, with an operator display, with pressure adjustment means and with a control panel allow an operator to actively monitor the status and position of the prosthetic socket casting cylinder apparatus and to determine and calculate a ratio of weight simultaneously exerted on the scale and on the socket.

A prosthetic socket casting cylinder and method wherein the inflatable distal interface has the configuration of a doughnut.

A prosthetic socket casting cylinder and method wherein the inflatable distal interface has the configuration of a ball.

A prosthetic socket casting cylinder and method for forming a prosthetic limb socket about a distal end portion of residual limb, while the residual limb is weight bearing, the prosthetic socket casting cylinder comprising: a tubular cylinder 15 defining an interior chamber 16 and having a top end portion 17 defining a top orifice 21 surrounded by a top peripheral edge 22 and a bottom end portion 18, the interior chamber 16 having an interior diameter 23 and an interior depth 24 that extends between the top end portion 17 and the bottom end portion 18, the cylinder 15 further having an outwardly facing surface 19 and an inwardly facing surface 20 and the cylinder 15 defines a port that communicates between the outwardly facing surface 19 and the interior chamber 16, and a sleeve securing band 25 extends substantially about the outwardly facing surface 19 proximate the top peripheral edge 22, and a slip flange 30 extends substantially about the outwardly facing surface 19 proximate the bottom end portion 18, the slip flange 30 defining plural spacedly arrayed fastener holes 32; a generally planar cylinder base 33 at the bottom end portion 18 of the cylinder 15, the cylinder base 33 having opposing top 34 and bottom 35 surfaces, and a peripheral edge 38, the cylinder base 33 has an exterior diametric dimension 39 that is larger than an exterior diametric dimension 37 of the cylinder 15, and the cylinder base 33 defines plural spacedly arrayed holes for fasteners 31 to extend therethrough to communicate with the plural spacedly arrayed fastener holes 32 defined in the slip flange 30 to secure the cylinder 15 to cylinder base 33, and the cylinder base 33 defines a hole 36 that communicates with the interior chamber 16; a scale 124 positioned beneath the cylinder base 33 and adjacent the bottom surface 35 thereof to sense downward pressure/weight exerted upon the cylinder 15, and the scale 124 is positioned upon a cylinder lift assembly 40; the cylinder lift assembly 40 supports the scale 124 and cylinder 15 and vertically adjustably positions the scale 124 and the cylinder 15 and the cylinder base 33 relative to an underlying supporting surface 137 and, the cylinder lift assembly 40 has, a generally planar base platform 41 that has a top surface 42 and an opposing bottom surface, and the base platform 41 carries plural spacedly arrayed post supports 53A that are positioned on the top surface 42 of the base platform 41 spacedly adjacent the scale 124, and each of the plural spacedly arrayed post supports 53A carries a follower bushing that is axially movable along a length of a threaded vertical adjustment post 43; plural spacedly arrayed threaded vertical adjustment posts 43, each of the plural spacedly arrayed threaded vertical adjustment posts 43 has a top end portion 45 and a bottom end portion 44 for contacting the underlying supporting surface 137, and each of the plural spacedly arrayed threaded vertical adjustment posts 43 communicates with one of the plural spacedly arrayed post supports 53A and the follower bushing 53 carried thereby, and each of the plural spacedly arrayed threaded vertical adjustment posts 43 extends generally perpendicularly to the base platform 41 and axially through the follower bushing 53, and a slave sprocket 48 is threadably interconnected to each of the plural spacedly arrayed threaded vertical adjustment post 53A so that the slave sprocket 48 rotates about and moves axially along the interconnected threaded vertical adjustment post 43, and a drive sprocket 49 is rotatably carried by the base platform 41 spacedly adjacent one slave sprocket 48 and a drive belt 50 operatively communicates between the drive sprocket 49 and the spacedly adjacent slave sprocket 48 so that rotation of the drive sprocket 49 is communicated to the slave sprocket 48 by the drive belt 50, and a drive means 56 operatively communicating with the drive sprocket 49 to rotate the drive sprocket 49 and to responsively cause the drive belt 50 to move and to responsively cause the slave sprocket 48 to rotate and to further responsively cause the slave sprocket's 48 and follower bushings 53 to move axially along the plural spacedly arrayed threaded vertical adjustment posts 43 which responsively causes each of the plural spacedly arrayed adjustment posts 43 to move axially upwardly/downwardly relative to the base platform 41 and relative to the underlying supporting surface 137; a proximal limiter 60 that substantially fluidically seals the top orifice 21 of the interior chamber 16; a distal interface cup assembly 90 within the interior chamber 16 of the cylinder 15, and the distal interface cup assembly 90 has an upper end portion 104 and a lower end portion 105, and has a platform 91 with an upper surface 95a and an opposing bottom surface 95b, and the platform 91 is vertically movable within the interior chamber 16 of the cylinder 15, and the platform 91 is carried at the upper end portion 104 of the distal interface cup assembly 90, and a distal interface socket 92 defining a concave depression having a known radius 93 is carried on the upper surface 95a of the platform 91, and the distal interface socket 92 is defined between plural spaced apart angulated side walls 94, and a vertically expandable/retractable lift means 100 communicates between the bottom surface 95b of the platform 91 and the top surface 34 of the cylinder base 33 within the interior chamber 16 of the cylinder 15 so that the lift means 100 controllably vertically adjustably moves and positionally maintains the platform 91 and the distal interface socket 92 within the interior chamber 16 of the cylinder 15 at a predetermined desired position, and a hole 96 is defined in the platform 91 and the hole 96 communicates between the distal interface socket 92 and the bottom surface 95b, and a load cell 99 positioned between the distal interface socket 92 and the upper surface 95a of the platform 91 to sense an amount of weight exerted upon the socket 92; an inflatable distal interface 110 removably positioned within the distal interface socket 92 to provide a spacer within the socket 92, the distal interface 110 having an upper surface 111, a bottom surface 112, an outer circumferential edge 113 and defines a medial hole 114, the distal interface 110 further having a port 117 for inflow and/or outflow of fluid to inflate/deflate the inflatable distal interface 110; a flexible sleeve 70 within the interior chamber 16 of the cylinder 15 that extends over and about the distal interface cup assembly 90 and over and about the inflatable distal interface 110, and the flexible sleeve 70 is formed of a resilient, fluid impermeable, elastomeric material and has a top end portion 73 and a bottom end portion 76 and defines a channel 75 extending between the top end portion 73 and the bottom end portion 76, the flexible sleeve 70 further having an outer surface 71 and an opposing inner surface 72, and the top end portion 73 defines a top peripheral edge 74 that is releasably secured to the top perimeter edge 22 of the top orifice 21 of the cylinder 15, and the bottom end portion 76 defines a bottom peripheral edge 77 that is releasably secured about the bottom end portion 18 of the cylinder 15 and to the top surface 34 of the cylinder base 33 by the slip flange 30; an air pump 120 pneumatically communicating with the interior chamberl6 of the cylinder 15 to pressurize the interior chamber 16 between the inner circumferential surface 20 of the cylinder 15 and the outer surface 72 of the flexible sleeve 70; a distal interface air pump 121 pneumatically communicating with the inflatable distal interface 110 to controllably inflate/deflate to the inflatable distal interface 110; a scale 125 placed upon an underlying supporting surface 137 adjacent to the base platform 41 to measure a weight that is not exerted upon the distal interface socket 92 of the distal interface cup assembly 90; an air pressure gauge 136 pneumatically communicating with the interior chamber 16 of the cylinder 15 to monitor air pressure within the interior chamber 16 in the space between the inner circumferential surface 20 and the outer surface 71 of the flexible sleeve 70; a controller 128 operatively communicating with the drive means 56 for vertically adjustably positioning the height of the cylinder 15 relative to a predetermined desired position, with the vertically expandable lift means 100 for vertically positioning the height of the distal interface socket 92 and platform 91 within the interior chamber 16 of the cylinder 15 relative to a predetermined desired position, with the load cell 99 to determine the amount of weight exerted upon the distal interface socket 92, with the air pump 120, with the scale 125, with a bleed valve operatively communicating with the bleed valve port 26, with the pressure relief valve operatively communicating with the pressure relief port 28, with an air pressure gauge 136 operatively communicating with an air pressure gauge port 29, with a distal interface air pump 121, with an operator display 129, with pressure adjustment means 131 and with a control panel 130 allow an operator to actively monitor the status and position of the prosthetic socket casting cylinder apparatus and to determine and calculate a ratio of weight exerted on the scale 125 and on the socket 92.

A prosthetic socket casting cylinder and method wherein the proximal limiter releasably engages with the top peripheral edge of the top orifice of the cylinder with a fastener, and the proximal limiter simultaneously releasably engages with an external surface of the residual limb spaced apart from a distal end of the residual limb, the proximal limiter further having an outer circumferential surface that is proximate to the top end portion of the cylinder and an inner circumferential surface that is variable in diameter and is proximate to the residual limb.

A prosthetic socket casting cylinder and method further comprising: a limb sleeve that is placed over and about the distal end portion of the residual limb; and casting material positioned over and about the limb sleeve, and wherein the casting material consolidates/hardens to form the prosthetic socket.

A method for forming a prosthetic limb socket about a distal end portion of residual limb while the residual limb is weight bearing, the method of forming the prosthetic socket comprising the steps: determining a total weight of a patient upon whose residual limb the prosthetic socket is to be formed; determining a height of the prosthetic socket; providing a cylinder defining an interior chamber having a top end portion, and a bottom end portion, the cylinder further having an outwardly facing surface and an inwardly facing surface and the cylinder defines a port that communicates between the outwardly facing surface and the interior chamber, and a slip flange extends substantially about the outwardly facing surface proximate the bottom end portion; providing a cylinder base secured to the bottom end portion of the cylinder, the cylinder base having a peripheral edge that has an exterior diametric dimension larger than an exterior diametric dimension of the cylinder; providing a scale positioned beneath the cylinder base to sense downward pressure/weight exerted upon the cylinder, and the scale is supported upon a cylinder lift assembly; providing a cylinder lift assembly having, a base platform with opposing top and bottom surfaces and carries plural spacedly arrayed post supports that are positioned on the top surface of the base platform and spacedly adjacent the scale, and each of the post supports carries a follower bushing that is axially movable along a length of the threaded vertical adjustment post, and each threaded vertical adjustment post threadably carries a slave sprocket that is axially rotatably movable along the length of the adjustment post, and each of the plural threaded vertical adjustment posts extend perpendicularly relative to the top and bottom surfaces of the platform and a bottom end portion of each threaded vertical adjustment post engages with the underlying supporting surface, and a drive belt operatively communicating between the slave sprockets so that rotation of one slave sprocket is communicated to a second slave sprocket by the drive belt, and a drive means operatively communicating with the drive belt; a distal interface cup assembly within the interior chamber of the cylinder, and the distal interface cup assembly has, an upper end portion, a lower end portion, and a platform that is vertically movable within the interior chamber, and the platform is carried at the upper end portion of the distal interface cup assembly, and a distal interface socket defining a concave depression in the upper surface of the platform, and a vertically expandable/retractable lift means operatively communicates between the platform and the cylinder base within the interior chamber to controllably vertically adjustably move and positionally maintain the platform within the interior chamber of the cylinder at a predetermined desired position, and a load cell between the socket and the platform to sense an amount of weight exerted upon the socket, and a distal interface within the distal interface socket; providing a flexible sleeve within the interior chamber of the cylinder that extends over and about the distal interface cup assembly and over and about the distal interface; providing an air pump pneumatically communicating with the interior chamber between the inwardly facing surface of the cylinder and an outwardly facing surface of the flexible sleeve; providing a scale to sense downward pressure/weight that is not exerted upon the distal interface socket; providing a controller operatively communicating with the drive means, with the vertically expandable/retractable lift means, with the load cell, with the air pump, with the scale, with an operator display, with adjustment means and with a control panel allow an operator to actively monitor the status and position of the prosthetic socket casting cylinder apparatus; monitoring, with the controller, the total weight of the patient, the weight exerted on the scale and the weight exerted on the casting cylinder and adjusting the pressure within the interior chamber, with the controller, so that the weight exerted by the patient upon the casting cylinder is a predetermined ratio of the total weight of the patient.

In compliance with the statute, the invention has been described in language more or less specific as to the structural and methodological features. It should be understood, however, that the invention is not limited to the specific features shown and described herein, since the means herein disclosed comprise preferred forms of putting the invention into effect. The invention is, therefore, claimed in any of its forms or modifications within the scope of the appended Claims appropriately interpreted in accordance with the Doctrine of Equivalence.

We claim:

1. A prosthetic socket casting cylinder and method for forming a prosthetic limb socket about a distal end portion of residual limb, while the residual limb is weight bearing, the prosthetic socket casting cylinder comprising:
a cylinder defining an interior chamber having a top end portion, and a bottom end portion, the cylinder further having an outwardly facing surface and an inwardly facing surface and the cylinder defines a port that communicates between the outwardly facing surface and the interior chamber, and a slip flange extends substantially about the outwardly facing surface proximate the bottom end portion;
a cylinder base secured to the bottom end portion of the cylinder, the cylinder base having a peripheral edge that has an exterior diametric dimension larger than an exterior diametric dimension of the cylinder;
a scale positioned beneath the cylinder base to sense downward pressure/weight exerted upon the cylinder, and the scale is supported upon a cylinder lift assembly;
the cylinder lift assembly has,
a base platform with opposing top and bottom surfaces and carries plural spacedly arrayed post supports that are positioned on the top surface of the base platform and spacedly adjacent the scale, and each of the post supports carries a follower bushing that is axially movable along a length of the threaded vertical adjustment post, and each threaded vertical adjustment post threadably carries a slave sprocket that is axially rotatably movable along the length of the adjustment post, and each of the plural threaded vertical adjustment posts extend perpendicularly relative to the top and bottom surfaces of the platform and a bottom end portion of each threaded vertical adjustment post engages with the underlying supporting surface, and
a drive belt operatively communicating between the slave sprockets so that rotation of one slave sprocket is communicated to a second slave sprocket by the drive belt, and
a drive means operatively communicating with the drive belt;
a distal interface cup assembly within the interior chamber of the cylinder, and the distal interface cup assembly has,
an upper end portion, a lower end portion, and a platform that is vertically movable within the interior chamber, and the platform is carried at the upper end portion of the distal interface cup assembly, and
a distal interface socket defining a concave depression in the upper surface of the platform, and
a vertically expandable/retractable lift means operatively communicates between the platform and the cylinder base within the interior chamber to controllably vertically adjustably move and positionally maintain the platform within the interior chamber of the cylinder at a predetermined desired position, and
a load cell between the socket and the platform to sense an amount of weight exerted upon the socket, and
a distal interface within the distal interface socket;
a flexible sleeve within the interior chamber of the cylinder that extends over and about the distal interface cup assembly and over and about the distal interface, the flexible sleeve having a top end portion that is positionally anchored to the cylinder, and a bottom end portion that is positionally secure to the cylinder base;
an air pump pneumatically communicating with the interior chamber between the inwardly facing surface of the cylinder and an outwardly facing surface of the flexible sleeve;
a scale to sense downward pressure/weight that is not exerted upon the distal interface socket;
a controller operatively communicating with the drive means, with the vertically expandable/retractable lift means, with the load cell, with the air pump, with the scale, with an operator display, with adjustment means and with a control panel allow an operator to actively monitor the status and position of the prosthetic socket casting cylinder apparatus and to determine and calculate a ratio of weight simultaneously exerted on the scale and on the socket.

2. The prosthetic socket casting cylinder and method of claim 1 and wherein the drive means is a motor.

3. The prosthetic socket casting cylinder and method of claim 1 and wherein the vertically expandable/retractable lift means is a telescoping cylinder.

4. The prosthetic socket casting cylinder and method of claim 1 and wherein the vertically expandable/retractable lift means is an expandable fluid containing bladder.

5. The prosthetic socket casting cylinder and method of claim 1 and further comprising:
a drive sprocket rotatably carried by the base platform and operatively communicating with the drive belt so that rotation of the drive sprocket is communicated to the slave sprocket.

6. The prosthetic socket casting cylinder and method of claim 5 and wherein the drive means rotates the drive sprocket which responsively moves the drive belt and which responsively rotates the slave sprockets to cause the cylinder base to move vertically relative to the underlying supporting surface.

7. The prosthetic socket casting cylinder and method of claim 1 and wherein the cylinder is tubular and defines a top orifice surrounded by a top peripheral edge, and has a bottom end portion, and the interior chamber has an interior diameter and an interior depth that extends between the top end portion and the bottom end portion, the tubular cylinder further having an outwardly facing surface and an inwardly facing surface and the tubular cylinder defines an air inlet port, a pressure relief port, a bleed valve port, an air pressure gauge port that all communicate between the outwardly facing surface and the interior chamber.

8. The prosthetic socket casting cylinder and method of claim 1 and further comprising:
a sleeve securing band that extends substantially about the outwardly facing surface of the cylinder proximate the top peripheral edge;
a slip flange that extends substantially about the outwardly facing surface of the cylinder proximate the bottom end portion, the slip flange defining plural spacedly arrayed fastener holes; and
the cylinder base defines plural spacedly arrayed holes for fasteners to extend therethrough to communicate with the plural spacedly arrayed fastener holes defined in the slip flange.

9. The prosthetic socket casting cylinder and method of claim 1 and further comprising:
a proximal limiter that substantially fluidically seals the top orifice of the interior chamber.

10. The prosthetic socket casting cylinder and method of claim 1 and wherein the distal interface socket has a known radius and is defined between plural spaced apart angulated side walls that extend from the upper surface of the platform.

11. The prosthetic socket casting cylinder and method of claim 1 and further comprising:
a hole is defined in the platform and the hole communicates between the distal interface socket and the bottom surface of the platform.

12. The prosthetic socket casting cylinder and method of claim 1 and wherein the distal interface is inflatable, and the distal interface has an upper surface, a bottom surface, an outer circumferential edge and defines a medial hole, the distal interface further has a port for inflow and/or outflow of fluid to inflate/deflate the inflatable distal interface.

13. The prosthetic socket casting cylinder and method of claim 1 and wherein the flexible sleeve is formed of a resilient, fluid impermeable, elastomeric material and has a top end portion and a bottom end portion and defines a channel extending between the top end portion and the bottom end portion, and the flexible sleeve further has an outwardly facing surface and an opposing inwardly facing surface, and the top end portion defines a top peripheral edge that is releasably secured to the top peripheral edge of the top orifice of the cylinder, and the bottom end portion defines a bottom peripheral edge that is releasably secured about the bottom end portion of the cylinder and to the top surface of the cylinder base.

14. The prosthetic socket casting cylinder and method of claim 1 and wherein the scale is placed upon an underlying supporting surface adjacent to the base platform to simultaneously measure weight that is not exerted upon the distal interface socket of the distal interface cup assembly.

15. The prosthetic socket casting cylinder and method of claim 1 and further comprising:
a controller operatively communicating with the drive means for vertically adjustably positioning and maintaining the cylinder relative to a predetermined desired position, with the vertically expandable/retractable lift means for vertically positioning the distal interface socket within the interior chamber of the cylinder relative to a predetermined desired position, with the load cell to determine an amount of weight exerted upon the distal interface socket, with the air pump, with the scale, with measuring instruments operatively communicating with the plural ports defined in the cylinder, with a distal interface air pump, with an operator display, with pressure adjustment means and with a control panel allow an operator to actively monitor the status and position of the prosthetic socket casting cylinder apparatus and to determine and calculate a ratio of weight simultaneously exerted on the scale and on the socket.

16. The prosthetic socket casting cylinder and method of claim 1, and wherein the inflatable distal interface has the configuration of a doughnut.

17. The prosthetic socket casting cylinder and method of claim 1, and wherein the inflatable distal interface has the configuration of a ball.

18. A prosthetic socket casting cylinder and method for forming a prosthetic limb socket about a distal end portion of residual limb, while the residual limb is weight bearing, the prosthetic socket casting cylinder comprising:
a tubular cylinder defining an interior chamber and having a top end portion defining a top orifice surrounded by a top peripheral edge and a bottom end portion, the interior chamber having an interior diameter and an interior depth that extends between the top end portion and the bottom end portion, the cylinder further having an outwardly facing surface and an inwardly facing surface and the cylinder defines a port that communicates between the outwardly facing surface and the interior chamber, and a sleeve securing band extends substantially about the outwardly facing surface proximate the top peripheral edge, and a slip flange extends substantially about the outwardly facing surface proximate the bottom end portion, the slip flange defining plural spacedly arrayed fastener holes;
a generally planar cylinder base at the bottom end portion of the cylinder, the cylinder base having opposing top and bottom surfaces, and a peripheral edge, the cylinder base has an exterior diametric dimension that is larger than an exterior diametric dimension of the cylinder, and the cylinder base defines plural spacedly arrayed holes for fasteners to extend therethrough to communicate with the plural spacedly arrayed fastener holes defined in the slip flange to secure the cylinder to cylinder base, and the cylinder base defines a hole that communicates with the interior chamber;
a scale positioned beneath the cylinder base and adjacent the bottom surface thereof to sense downward pressure/weight exerted upon the cylinder, and the scale is positioned upon a cylinder lift assembly;
the cylinder lift assembly supports the scale and cylinder and vertically adjustably positions the scale and the cylinder and the cylinder base relative to an underlying supporting surface and, the cylinder lift assembly has,
a generally planar base platform that has a top surface and an opposing bottom surface, and the base platform carries plural spacedly arrayed post supports that are positioned on the top surface of the base platform spacedly adjacent the scale, and each of the plural spacedly arrayed post supports carries a follower bushing that is axially movable along a length of a threaded vertical adjustment post;
plural spacedly arrayed threaded vertical adjustment posts, each of the plural spacedly arrayed threaded vertical adjustment posts has a top end portion and a bottom end portion for contacting the underlying supporting surface, and each of the plural spacedly arrayed threaded vertical adjustment posts communicates with one of the plural spacedly arrayed post supports and the follower bushing carried thereby, and each of the plural spacedly arrayed threaded vertical adjustment posts extends generally perpendicularly to the base platform and axially through the follower bushing, and
a slave sprocket is threadably interconnected to each of the plural spacedly arrayed threaded vertical adjustment post so that the slave sprocket rotates about and moves axially along the interconnected threaded vertical adjustment post, and
a drive sprocket is rotatably carried by the base platform spacedly adjacent one slave sprocket and a drive belt operatively communicates between the drive sprocket and the spacedly adjacent slave sprocket so that rotation of the drive sprocket is communicated to the slave sprocket by the drive belt, and
a drive means operatively communicating with the drive sprocket to rotate the drive sprocket and to responsively cause the drive belt to move and to responsively cause the slave sprocket to rotate and to further responsively cause the slave sprocket's and follower bushings to move axially along the plural spacedly arrayed threaded vertical adjustment posts which responsively causes each of the plural spacedly arrayed adjustment posts to move axially upwardly/downwardly relative to the base platform and relative to the underlying supporting surface;
a proximal limiter that substantially fluidically seals the top orifice of the interior chamber;
a distal interface cup assembly within the interior chamber of the cylinder, and the distal interface cup assembly has an upper end portion and a lower end portion, and has
a platform with an upper surface and an opposing bottom surface, and the platform is vertically movable within the interior chamber of the cylinder, and the platform is carried at the upper end portion of the distal interface cup assembly, and
a distal interface socket defining a concave depression having a known radius is carried on the upper surface of the platform, and the distal interface socket is defined between plural spaced apart angulated side walls, and
a vertically expandable/retractable lift means communicates between the bottom surface of the platform and the top surface of the cylinder base within the interior chamber of the cylinder so that the lift means controllably vertically adjustably moves and positionally maintains the platform and the distal interface socket within the interior chamber of the cylinder at a predetermined desired position, and
a hole is defined in the platform and the hole communicates between the distal interface socket and the bottom surface, and
a load cell positioned between the distal interface socket and the upper surface of the platform to sense an amount of weight exerted upon the socket;
an inflatable distal interface removably positioned within the distal interface socket to provide a spacer within the socket, the distal interface having an upper surface, a bottom surface, an outer circumferential edge and defines a medial hole, the distal interface further having a port for inflow and/or outflow of fluid to inflate/deflate the inflatable distal interface;
a flexible sleeve within the interior chamber of the cylinder 1 that extends over and about the distal interface cup assembly and over and about the inflatable distal interface, and the flexible sleeve is formed of a resilient, fluid impermeable, elastomeric material and has a top end portion and a bottom end portion and defines a channel extending between the top end portion and the bottom end portion, the flexible sleeve further having an outer surface and an opposing inner surface, and the top end portion defines a top peripheral edge that is releasably secured to the top perimeter edge of the top orifice of the cylinder, and the bottom end portion defines a bottom peripheral edge that is releasably secured about the bottom end portion of the cylinder and to the top surface of the cylinder base by the slip flange;
an air pump pneumatically communicating with the interior chamber of the cylinder to pressurize the interior chamber between the inner circumferential surface of the cylinder and the outer surface of the flexible sleeve;
a distal interface air pump pneumatically communicating with the inflatable distal interface to controllably inflate/deflate to the inflatable distal interface;
a scale placed upon an underlying supporting surface adjacent to the base platform to measure a weight that is not exerted upon the distal interface socket of the distal interface cup assembly;
an air pressure gauge pneumatically communicating with the interior chamber of the cylinder to monitor air pressure within the interior chamber in the space between the inner circumferential surface and the outer surface of the flexible sleeve;
a controller operatively communicating with the drive means for vertically adjustably positioning the height of the cylinder relative to a predetermined desired position, with the vertically expandable lift means for vertically positioning the height of the distal interface socket and platform within the interior chamber of the cylinder relative to a predetermined desired position, with the load cell to determine the amount of weight exerted upon the distal interface socket, with the air pump, with the scale, with a bleed valve operatively communicating with the bleed valve port, with the pressure relief valve operatively communicating with the pressure relief port, with an air pressure gauge operatively communicating with an air pressure gauge port, with a distal interface air pump, with an operator display, with pressure adjustment means and with a control panel allow an operator to actively monitor the status and position of the prosthetic socket casting cylinder apparatus and to determine and calculate a ratio of weight exerted on the scale and on the socket.

19. The prosthetic socket casting cylinder and method of claim 18, and wherein the proximal limiter releasably engages with the top peripheral edge of the top orifice of the cylinder with a fastener, and the proximal limiter simultaneously releasably engages with an external surface of the residual limb spaced apart from a distal end of the residual limb, the proximal limiter further having an outer circumferential surface that is proximate to the top end portion of the cylinder and an inner circumferential surface that is variable in diameter and is proximate to the residual limb.

20. The prosthetic socket casting cylinder and method of claim 18, and further comprising:
a limb sleeve that is placed over and about the distal end portion of the residual limb; and
casting material positioned over and about the limb sleeve, and wherein the casting material consolidates/hardens to form the prosthetic socket.

21. A method for forming a prosthetic limb socket about a distal end portion of residual limb while the residual limb is weight bearing, the method of forming the prosthetic socket comprising the steps:
determining a total weight of a patient upon whose residual limb the prosthetic socket is to be formed;
determining a height of the prosthetic socket;
providing a cylinder defining an interior chamber having a top end portion, and a bottom end portion, the cylinder further having an outwardly facing surface and an inwardly facing surface and the cylinder defines a port that communicates between the outwardly facing surface and the interior chamber, and a slip flange extends substantially about the outwardly facing surface proximate the bottom end portion;
providing a cylinder base secured to the bottom end portion of the cylinder, the cylinder base having a peripheral edge that has an exterior diametric dimension larger than an exterior diametric dimension of the cylinder;
providing a scale positioned beneath the cylinder base to sense downward pressure/weight exerted upon the cylinder, and the scale is supported upon a cylinder lift assembly;
providing a cylinder lift assembly having,
a base platform with opposing top and bottom surfaces and carries plural spacedly arrayed post supports that are positioned on the top surface of the base platform and spacedly adjacent the scale, and each of the post supports carries a follower bushing that is axially movable along a length of the threaded vertical adjustment post, and each threaded vertical adjustment post threadably carries a slave sprocket that is axially rotatably movable along the length of the adjustment post, and each of the plural threaded vertical adjustment posts extend perpendicularly relative to the top and bottom surfaces of the platform and a bottom end portion of each threaded vertical adjustment post engages with the underlying supporting surface, and
a drive belt operatively communicating between the slave sprockets so that rotation of one slave sprocket is communicated to a second slave sprocket by the drive belt, and
a drive means operatively communicating with the drive belt;
a distal interface cup assembly within the interior chamber of the cylinder, and the distal interface cup assembly has,
an upper end portion, a lower end portion, and a platform that is vertically movable within the interior chamber, and the platform is carried at the upper end portion of the distal interface cup assembly, and
a distal interface socket defining a concave depression in the upper surface of the platform, and
a vertically expandable/retractable lift means operatively communicates between the platform and the cylinder base within the interior chamber to controllably vertically adjustably move and positionally maintain the platform within the interior chamber of the cylinder at a predetermined desired position, and
a load cell between the socket and the platform to sense an amount of weight exerted upon the socket, and
a distal interface within the distal interface socket;
providing a flexible sleeve within the interior chamber of the cylinder that extends over and about the distal interface cup assembly and over and about the distal interface;
providing an air pump pneumatically communicating with the interior chamber between the inwardly facing surface of the cylinder and an outwardly facing surface of the flexible sleeve;
providing a scale to sense downward pressure/weight that is not exerted upon the distal interface socket;
providing a controller operatively communicating with the drive means, with the vertically expandable/retractable lift means, with the load cell, with the air pump, with the scale, with an operator display, with adjustment means and with a control panel allow an operator to actively monitor the status and position of the prosthetic socket casting cylinder apparatus;
monitoring, with the controller, the total weight of the patient, the weight exerted on the scale and the weight exerted on the casting cylinder and adjusting the pressure within the interior chamber, with the controller, so that the weight exerted by the patient upon the casting cylinder is a predetermined ratio of the total weight of the patient.
